(19) United States
(12) Patent Application Publication
(10) Pub. No.: US 2008/0306453 A1
(43) Pub. Date: Dec. 11, 2008

(54) Coupling wire guide and method for making same

(75) Inventors: James C. Elsesser, Bloomington, IN (US); Don Patterson, Bloomington, IN (US); Jakob A. Richart, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/810,614

(22) Filed: Jun. 6, 2007

(51) Int. Cl. A61M 25/09

(52) U.S. Cl. 604/264; 600/585

(57) ABSTRACT

A coupling wire guide and an improved method for bonding a mandrel to a tracking tip in a reliable and long-lasting manner are provided. The coupling wire guide includes a mandrel connected to a tracking tip. The mandrel includes a distal portion, including first and second portions integral to the mandrel, the second portion being wider than the first portion. The tracking tip includes a first tubular section and a second tubular section adapted to receive a previously introduced wire guide therein. The first tubular section is disposed over and bonded to the first and second portions to form the coupling wire guide. A coil may be attached to the distal portion upstream of the second portion. In a preferred embodiment, the second portion is a spherical structure adhesively bonded to the first tubular section. Use of a medical grade adhesive in conjunction with the second portion has been surprisingly found to increase tensile joint strength and decrease tensile joint strength variability.

### TECHNICAL FIELD

This invention relates generally to a wire guide for use in percutaneous interventional procedures, and more particularly, a method for making a wire guide that can be coupled to a previously introduced wire guide for assistance during interventional procedures in vessels with proximal tortuosity, or as a more substantial wire guide for angioplasty procedures, stenting procedures, and other device placement procedures and their related devices.

### BACKGROUND

Wire guides are typically used to navigate the vasculature of a patient during intracorporeal procedures. Once the wire guide has been introduced, it may then be used to introduce one or more medical catheter devices. Many conventional wire guides are typically 0.014 inches in diameter and have a lubricous coating to enhance wire guide introduction movement. These conventional “floppy” wire guides have sufficient flexibility and torque control for navigation through tortuous vessels. In certain procedures or situations, it is desirable to enhance the conventional wire guide with a supplemental wire guide. The supplemental wire guide is placed alongside the conventional wire guide, for example, to straighten out the vessel curves and facilitate further wire guide movement. The supplemental wire guide provides additional support and enhances the tracking of balloons, stents, stent delivery devices, atherectomy devices, and other medical catheter devices. This technique is commonly referred to as the “Buddy Wire” technique, details of which are disclosed in U.S. patent application Ser. No. 11/081,146, filed Mar. 16, 2005, which is expressly incorporated by reference herein.

Several unique supplemental wire guides have been developed which are structured to be slidably coupled to a conventional wire guide (or any previously introduced wire guide) to provide easy and reliable navigation through the vasculature to a position proximate the previously introduced wire guide. These supplemental wire guides are commonly referred to as coupling wire guides or Buddy Wires. Exemplary coupling wire guides are disclosed in U.S. Provisional Patent Application Nos. 60/711,102 filed Aug. 25, 2005; 60/711,261 filed Aug. 25, 2005; 60/763,511 filed Jan. 31, 2006; and 60/763,523 filed Jan. 31, 2006, the disclosures of which are hereby incorporated herein by reference in their entireties. Although other variations exist, a coupling wire guide generally includes a separately formed tracking tip that is joined to a distal portion of the coupling wire guide and is configured to be slidably coupled to the conventional wire guide.

Typically, the tracking tip in a coupling wire guide is made from a nickel-titanium alloy, such as Nitinol, which has favorable properties of strength, light weight, superelasticity, and shape memory. Because Nitinol is somewhat expensive, its use in medical devices is often limited to where it is most needed. Thus, less expensive biocompatible metals, like stainless steel, are often utilized for less-critical elements. Unfortunately, one of the limitations associated with nickel-titanium alloys, such as Nitinol, is the difficulty in stably bonding it to other materials, such as stainless steel, that form the distal portion of the coupling wire. Accordingly, there exists a need to provide a coupling wire guide and a method for stable, reproducible bonding of a tracking tip to the coupling wire guide.

### SUMMARY

The present invention provides a coupling wire guide for coupling to a previously introduced wire guide that is more easily, reliably, and stably constructed. In one aspect, the coupling wire guide includes a mandrel connected to a tracking tip. The mandrel includes a distal portion, including first and second portions integral to the mandrel, whereby the second portion is wider in diameter compared to the first portion. The tracking tip includes a first tubular section and a second tubular section adapted to receive a previously introduced wire guide therein. The first tubular section is disposed over and bonded to the first and second portions. A coil may be attached to the distal portion upstream of the second portion. In a preferred embodiment, the second portion is a spherical structure at the terminal end of the mandrel, which is adhesively bonded to the inside of the first tubular section. Use of a medical grade adhesive in conjunction with the second portion has been surprisingly found to increase tensile joint strength and decrease tensile joint strength variability.

In another aspect, a method for forming the above described coupling wire guide includes disposing the first tubular section of the tracking tip over the distal portion of the wire guide and bonding the first tubular section to the first and second portions to form the coupling wire guide. In a preferred embodiment, a medical grade adhesive is applied to the second portion to facilitate a more secure, reproducible attachment to the tracking tip. As the adhesive becomes cured, the distal portion of the mandrel locks the mandrel into the tracking tip, thereby creating a secure joint between the main body and the tracking tip.

### BRIEF DESCRIPTION OF THE DRAWINGS

The accompanying drawings incorporated in and forming a part of the specification illustrate several aspects of the present invention, and together with the description serve to explain the principles of the invention. In the drawings:

FIG. 1 is a side view of a coupling wire guide in accordance with the present invention;

FIG. 2A is an enlarged side view, partially in cross-section, of the coupling wire guide depicted in FIG. 1.

FIG. 2B is a cross-sectional view, taken about the line 2B-2B in FIG. 2A;

FIGS. 3A and 3B are side views of a proximal coupling wire guide portion depicting a further aspect of the present invention.

### DETAILED DESCRIPTION

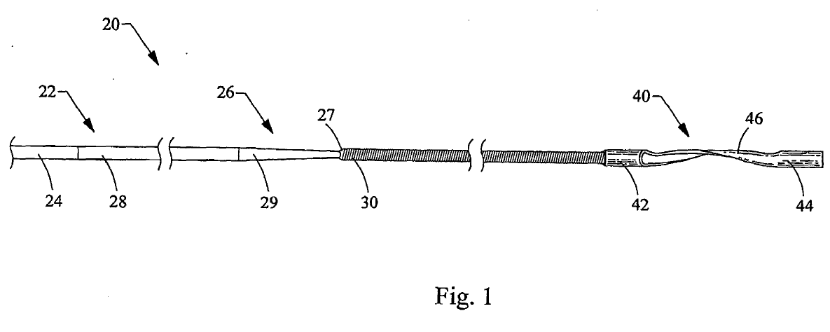

Turning now to the drawings, FIG. 1 depicts an exemplary coupling wire guide 20 in accordance with the present invention. The coupling wire guide 20 includes a mandrel 28 having main body 22, including a proximal portion 24 and a distal portion 26. The distal portion 26 is connected to a tracking tip 40. As depicted in FIG. 1, the distal portion 26 may include a tapered, reduced diameter section 29.

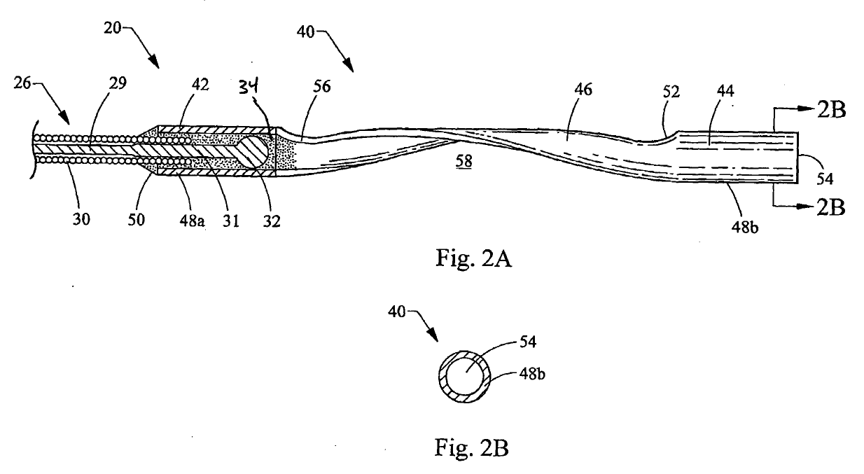

As shown in FIG. 2A, the distal portion 26 of the mandrel 28 includes a first portion 31 integrally connected to a second portion 32. The second portion 32 has a wider diameter than the first portion 31. The first portion 31 and the second portion 32 define a distal end portion of the main body 22, portions of which are configured for binding to inside surfaces of a proximal portion 42 of the tracking tip 40.

In FIG. 2A, the first portion 31 has a greater diameter than the reduced diameter section 29. However, the first portion 31 may also have a diameter equivalent to or tapered relative to the reduced diameter section 31. In addition, the second portion 32 is preferably disposed at the terminal end of the mandrel 28. Alternatively, the second portion 32 may be positioned elsewhere within the distal portion 26 of the mandrel 28 so long as it is capable of bonding to inside surfaces of a proximal portion 42 of the tracking tip 40.

As shown in FIG. 2A, a flexible helical coil 30 may be disposed over the distal portion 26 of the mandrel, preferably over the portions of the reduced diameter section 29 and the first portion 31. The distal end of the coil 30 is preferably spaced apart from the second portion 32. The first portion 31 may extend beyond the coil 30 to expose an integrally formed widened end portion 32.

According to the present invention, the second portion 32 is configured for bonding to the inside of the proximal portion 42 of the tracking tip 40 to provide greater joint strength between the main body 22 and the tracking tip 40, as described in greater detail below. As shown in FIG. 2A, the second portion 32 can be shaped into a ball, slidably fitting into a proximal portion 42 of the tracking tip 40. The second portion 32 may be integrally formed at the end of the mandrel 28 by contacting the distal end of the mandrel 28 with a suitable welder. For example, it is believed that plasma, laser, tungsten inert gas (TIG), or other suitable welding can be used to integrally form a second portion 32 on the distal end of the mandrel 28 that is wider in diameter compared to the first portion 31. The second portion 32 may be formed into a variety of three-dimensional shapes slidably fitting the inner diameter of the proximal portion 42 of the tracking tip 40. Suitable shapes may include linear, curved, or curvilinear edges, and/or rectangular, square, circular or oval faces.

In the embodiment shown in FIG. 2A, the proximal portion 42 of the tracking tip 40 is formed as a tubular structure 48a having a proximal opening 50 sized to receive the ball-shaped mandrel portion 32. The tracking tip 40 further includes a coupling portion 44 defining a second tubular structure 48b configured to receive a previously introduced wire guide. The coupling portion 44 includes a circular proximal opening 52 and a distal opening 54 through which the proximal end of the previously introduced wire guide passes (FIG. 2B).

An interconnecting portion 46 of the tracking tip 40 interconnects the proximal and coupling portions 42, 44. The interconnecting portion 46 is formed as a circumferentially winding strip 56 that provides flexibility to the tracking tip 40 and provides an opening or open area 58 for receiving the previously introduced wire guide. The open area 58 is in communication with the circular proximal opening 52 of the coupling portion 44 through which the distal end of the previously introduced wire guide passes. Specifically, the strip 56 follows a curved path between the proximal and coupling portions 42, 44, and preferably follows a helical path between the proximal and coupling portions 42, 44. In this manner, the interconnecting portion 46 provides a degree of flexibility to the tracking tip 40, while maintaining sufficient rigidity to securely link the proximal and coupling portions 42, 44.

Components of the tracking tip 40, including the interconnecting portion 46 or strip 56, may be symmetrically configured to facilitate bonding of the distal portion 26 of the main body 22 through either opening 50, 54 of the tracking tip 40.

The length of the wire guide 20 may range from about 40 cm to about 480 cm. More preferably the wire guide may range in length from about 125 cm to about 250 cm. In a preferred embodiment, the wire guide 20 is about 180 cm in length.

The wire guide 20 may be made from a variety of different material components differing in size and shape. In a preferred embodiment, the wire guide 20 includes a mandrel 28 having a diameter between from about 0.006 to about 0.014 inches; an outer coil having an inner diameter between about 0.006 to about 0.007 and an outer diameter between about 0.013 to about 0.014 inches; and/or a laser cut, electropolished cannula 40 having a 0.0028 inch wall thickness and a length from about 5.0 mm to about 10.0 mm. Preferably, the reduced diameter portion 29 of the mandrel 28 extends longitudinally from about 1.8 cm to about 6.9 cm, while the wound coil 30 extends longitudinally from about 3.0 cm to about 6.0 cm. Also, the first tubular member 48a of the tracking tip 40 preferably overlaps with the distal portion 26 of the main body by about 0.5 mm to about 2.5 mm.

The mandrel 28 is preferably formed from a flexible, elastic, bendable, kink-resistant, sterilizable, biocompatible material having sufficient flexibility to traverse a patient's vasculature. Preferred materials include Nitinol and stainless steel. The choice of material may depend on factors such as cost and degree of stiffness required. Nitinol is preferred for applications requiring more flexibility; stainless steel may be preferred for applications where greater stiffness is required.

In one embodiment, the mandrel 28 is constructed from a Nitinol wire. The Nitinol wire may be formed by drawing through dies and then ground, preferably using a center-less grinding technique. Alternatively, the mandrel may be formed from a thin spring tempered stainless steel material.

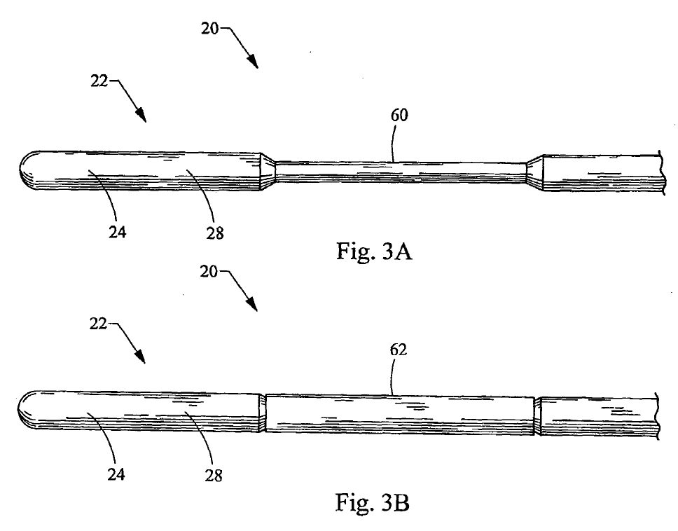

A variety of different sizes and types of mandrels known to those of skill in the art may be used. The mandrel 28 may have a substantially uniform diameter at the proximal end. Alternatively, as shown in FIGS. 3A and 3B, the mandrel 28 may have an additional reduced diameter section 60 toward the proximal end overlayed with a colored sleeve 62 to aid in identifying and distinguishing the wire guide 20 when performing percutaneous procedures involving a plurality of different wire guides (see below).

The outside diameter of the mandrel 28 may range from about 0.004 inches to about 0.04 inches. Preferably the outside diameter of the mandrel 28 ranges from 0.0025 inches to about 0.05 inches. More preferably the outside diameter of the mandrel ranges from about 0.005 inches to about 0.02 inches. In a particular embodiment the mandrel includes a substantially uniform diameter of about 0.0136 inches at the proximal end and a reduced diameter of about 0.0065 inches toward the distal end.

A proximal end of the coil 30 is joined to a distal portion of the mandrel 28 as illustrated in FIGS. 1 and 2A. The coil 30 may be joined at its proximal end to the mandrel 28 by a solder joint 27, adhesive, or any other suitable bonding means known to those of ordinary skill in the art. As used in the present disclosure, the ‘term “solder” is used to denote a direct fusion between a first metal material and a dissimilar, metal solder material. The term “adhesive” is used to denote a substance, such as a polymeric material, which is employed to bond two separate materials together, but does not include a solder as such.

The coil 30 may be radiopaque and made of stainless steel, platinum, platinum-nickel, iridium, palladium, tantalum, tungsten, or alloys thereof; nickel-titanium alloy, such as Nitinol; or any other suitable material known to those of skill in the art. The coil 30 also may be rendered more radiopaque by coating, for example, a stainless steel coil with gold or other known radiopaque marker materials as described below. Preferably, at least a portion of the coil 30 is made of a radiopaque metal to facilitate fluoroscopic imaging while inside a patient's body. The coil may be formed by drawing a wire though dies and pulling the wire across a stress-inducing surface to form a coil shape by or by winding the Nitinol wire around the mandrel 28 and then heat setting the Nitinol wire.

Radiopaque marker materials may be added to one or more components of the wire guide 20 to facilitate fluoroscopic imaging. In particular, radiopaque materials, fillers, marker bands or powders may be associated with one or more of the mandrel 28, coil 30, tracking tip 40, or any component part thereof. For example, a radiopaque marker may be incorporated into the tracking tip 40 by gold plating the terminal 1.0-1.5 mm of the coupling portion 44.

Exemplary radiopaque marker materials include but are not limited to, platinum, palladium, iridium, gold, tungsten, tantalum, tantalum powder, bismuth, bismuth oxychloride, barium, barium sulphate, iodine, alloys thereof, and the like. The radiopaque materials can be incorporated in the wire guide by a variety of common methods, such as adhesive bonding, lamination between two material layers, vapor deposition, including the materials and methods described in U.S. 2003/0206860, the disclosure of which is incorporated herein by reference.

The tracking tip 40 typically is formed from Nitinol, which can be formulated to provide superelasticity and/or shape memory. The tracking tip 40 preferably is constructed from a Nitinol cannula as described in U.S. Pat. Nos. 4,759,487 and 4,852,790, the disclosures of which are incorporated by reference herein. Alternatively, the cannula may be constructed by drawing Nitinol stock seamlessly, or by drawing, rolling and welding. Preferably, the cannula is laser-cut to shape, electropolished and/or media-blasted and/or abrasive ground to the desired surface finish, and heat-treated to obtain the desired properties.

The Nitinol materials used in the present invention have an austenitic finish temperature (A_{f}) less than body temperature and in the range of 0 to 35 degrees C. This provides sufficient structural integrity as well as a very fast recovery time from stress-induced deformation. The A_{f} of the Nitinol materials may be below room temperature or it may be above room temperature and below body temperature. In a preferred embodiment, A_{f} is between about 5-10 degrees C. (below room temperature) to provide a fully austenitic material both outside and inside the patient. In another preferred embodiment, A_{f} is in the range of about 10-35 degrees C., to provide a partially austenitic material outside the patient to improve handling, while fully austenitic inside the patient.

As indicated above, the bonding of Nitinol to other materials presents unique and problematic manufacturing challenges. In one aspect, the present invention provides an improved method for bonding nickel-titanium-based materials. In particular, a medical grade adhesive 34 is preferably applied to the ball-shaped mandrel portion 32 of the mandrel 28 (see FIGS. 1, 2). The ball-shaped mandrel portion 32 is inserted through the proximal opening 50 of the tracking tip 40 within the first tubular section 48a and is positioned near the interconnecting portion 46. Through surface attraction and capillary action, the adhesive 34 forms a joint tapering away from the ball-shaped mandrel portion 32 toward the proximal opening 50 at one end and into the interconnecting portion 46 of the tracking tip 58 at the other end (FIG. 2A). Upon curing of the adhesive 34, the ball-shaped mandrel portion 32, spaced from the distal end of the coil 30, becomes stably locked to the mandrel 28 into the tracking tip 40. In this manner, a secure interconnection between the distal portion 26 of the main body 22 and the tracking tip 40 is provided, particularly between a stainless steel coil 30 and a Nitinol tracking tip 40, whereby the resulting joint is stronger and more reliable than a solder joint. In addition, the tapered adhesive portion near the proximal opening 50 reduces undesirable sharp edges.

A variety of medical grade adhesives may be used for bonding the mandrel 28 to the tracking tip 40. Preferably, the adhesive is a UV-curable adhesive designed for use in medical devices. Preferred UV-curable adhesives are solvent-free and cure upon exposure to UV light, visible light, and/or heat. To facilitate in-line quality control of adhesion, the adhesive may include suitable fluorescing agents or photoinitiators as described in U.S. Pat. No. 6,080,450. Suitable adhesives are non-toxic in a cured state and pass the biocompatibility tests in accordance with USP Class VI and/or ISO 10993 biocompatibility certification status.

Exemplary UV-curable adhesives include acrylates, cyanoacrylates, urethanes, urethane (meth)acrylates, epoxies and the like. The choice of adhesive may depend on a variety of factors, such as the type of materials bonded, nature of the structures bonded, and desired cured properties, including durometer hardness, tensile at break, elongation at break etc. Preferred adhesives include the CTH-series adhesives from Dymax Corporation (Torrington, Conn.), including 203A-CTH, 204-CTH, 206-CTH, 207-CTH, and 208-CTH.

Medical grade adhesives represent a preferred agent for bonding a Nitinol tracking tip 40 to the distal end of a coupling wire guide 20 containing a second portion 32. Other means for bonding the second portion 32 to the tracking tip 40 may include soldering, welding or any other or any other bonding means known to those of skill in the art.

Of course, it will be recognized by those skilled in the art that many different sizes and types of mandrels, coils, and tracking tips may be employed in conjunction with the present invention, including any of those disclosed in co-pending U.S. Provisional Patent Application Nos. 60/711,102 filed Aug. 25, 2005; 60/711,261, filed Aug. 25, 2005; 60/763,511, filed Jan. 31, 2006; 60/763,523, filed Jan. 31, 2006; and 60/815,805, filed Jun. 22, 2006, all of which are expressly incorporated by reference herein.

The coupling wire guide 20 of the present invention is typically coupled to a previously introduced wire guide outside of the patient's body. To help avoid confusion during percutaneous interventional procedures requiring multiple exchanges of wire guides, a further aspect of the present invention includes an identification section allowing a physician to readily distinguish between the proximal ends of the previously introduced wire guide(s) and the coupling wire guide of the present invention (FIGS. 3A, 3B).

In FIG. 3A, the coupling wire guide 20, including a proximal portion 24 of the main body 22, includes a wire guide identification section 60. In FIG. 3A, the identification section 60 corresponds to a ground down portion of the mandrel having a reduced diameter overlayed with a suitable colorized sleeve 62 (FIG. 3B) aiding in the identification of the coupling wire guide 20. Preferably, the reduced diameter section is ground down by about 0.002 to about 0.006 inches. The identification section 60 may encompass any length suitable for identification. Preferably the identification section ranges from about 2 to about 10 cm. Alternative wire guide identification configurations for use in the present invention are described in co-pending U.S. Provisional Patent Application No. 60/727,816, filed Oct. 18, 2005, the teachings of which are expressly incorporated by reference herein.

In an alternative embodiment, one or more portions of the mandrel are covered with an identifiable coating, preferably a lubricous polymer, such as polytetrafluoroethylene (Teflon), which has a colorizing finish (such as bright yellow). The coating may be applied to any portion of the mandrel, including ground down portions thereof. Suitable coating materials for use in the present invention are disclosed in co-pending U.S. patent application Ser. No. 10/227,048, filed Aug. 23, 2002 and Ser. No. 10/831,740, filed Apr. 23, 2004, which are expressly incorporated by reference herein.

### EXAMPLE

Tensile joint strengths of tracking tips bonded to a coiled Nitinol mandrel 28 with or without a second portion 32 according to FIG. 1 integrally formed thereon extending from the distal end of a stainless steel coil 30. In a first sample type, a ball-shaped mandrel portion 32 was spaced downsteam of the stainless steel coil 30 within the first tubular section 42. In a second sample type, a stainless steel coil 30 was attached to the terminal end of the Nitinol mandrel 28. The first sample type was bonded to the first tubular section 42 of the tracking tip 40 using adhesive bonding. A biocompatible, UV-curable medical grade adhesive (207-CTH Series MD® Medical Device adhesive, Dymax Corporation, Torrington, Conn.) was used as described above. The second sample type was bonded to the first tubular section 42 of the tracking tip 40 by soldering.

Surprisingly, the mean tensile strength of adhesively bonded joints in 41 samples of the first sample type was 17.3 Newtons (SD=0.9 N). By comparison, the mean tensile joint strength in 45 samples of the second sample type was 13.3 Newtons (SD=3.7 N). Thus, compared to ball-less soldering of coiled wire guide ends, weld ball/adhesive bonding resulted in a 30% increase in tensile joint strength (from 13.3 N to 17.3 N). In addition, variability in tensile joint strength dropped considerably, as evidenced by a 76% decrease in standard deviation (from 3.7 N to 0.9 N).

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A coupling wire guide for coupling to a previously introduced wire guide, the coupling wire guide comprising:
a mandrel including a distal portion, including a first portion and a second portion, wherein the first and second portions are integral to the mandrel and the second portion is wider than the first portion; and
a tracking tip connected to the distal portion, the tracking tip comprising a first tubular section and a second tubular section adapted for receiving the previously introduced wire guide therein;
wherein the first tubular section is disposed over and bonded to the first and second portions.

2. The coupling wire guide of claim 1, wherein the distal portion is adhesively bonded to the first tubular section.

3. The coupling wire guide of claim 1, wherein the tracking tip includes an interconnecting portion interconnecting the first and second tubular sections, the interconnecting portion including a strip extending axially and circumferentially in communication with a distal opening in the first tubular section and a proximal opening in the second tubular section.

4. The coupling wire guide of claim 1, wherein the second portion comprises a spherical structure at a terminal end of the mandrel.

5. The coupling wire guide of claim 1, wherein a coil is disposed over the first portion in the first tubular section.

6. The coupling wire guide of claim 5, wherein the coil is not disposed over part of the distal portion in the first tubular section.

7. The coupling wire guide of claim 5, wherein the coil extends over a third mandrel portion outside of the first tubular section, wherein the third mandrel portion has a diameter less than a first diameter of the first portion.

8. The coupling wire guide of claim 5, wherein the tracking tip is formed from Nitinol and the coil comprises a metal selected from the group consisting of from stainless steel, platinum, platinum-nickel, palladium, iridium, tantalum, tungsten, and alloys thereof.

9. The coupling wire guide of claim 1, wherein the mandrel comprises an identification sleeve disposed over a reduced diameter section of the mandrel.

10. The coupling wire guide of claim 1, wherein a colorized polymer coating is disposed over at least a portion of the mandrel.

11. The coupling wire guide of claim 1, wherein the tracking tip is made from Nitinol material having an A_{f} in the range of about 0 to 35 degrees Celsius.

12. A method of forming the coupling wire guide of claim 1, comprising the steps of:
disposing the first tubular section of the tracking tip over the distal portion of the mandrel; and
bonding the first tubular section to the first and second portions of the distal portion to form the coupling wire guide.

13. The method of claim 12, wherein the step of bonding comprises bonding with a medical grade adhesive.

14. The method of claim 12, wherein the step of bonding comprises soldering.

15. The method of claim 12, comprising the step of disposing a coil over the distal portion of the mandrel.

16. The method of claim 15, comprising the step of welding a spherical structure onto the distal end of the mandrel distal to the coil.

17. The method of claim 15, wherein the tracking tip is formed from Nitinol and the coil is formed from stainless steel or platinum.

18. The method of claim 12, comprising the step of grounding down a proximal portion of the mandrel and applying an identification sleeve thereto.

19. The method of claim 12, comprising the step of applying a colorized polymer coating to a proximal portion of the mandrel.

20. The method of claim 13, comprising application of the adhesive so that the tensile strength of a joint formed between a distal portion of the mandrel and the tracking tip is at least 17 newtons.

21. The method of claim 12, comprising the step of incorporating a radiopaque marker material into the tracking tip.

22. A coupling wire guide for coupling to a previously introduced wire guide, the coupling wire guide comprising:
a mandrel including a first mandrel portion and a second mandrel portion, the second mandrel portion comprising a distal mandrel end, wherein the first and second mandrel portions are integral to the mandrel and the second mandrel portion is wider than the first mandrel portion; and
a tracking tip comprising a proximal tubular section axially spaced from a distal tubular section, the proximal and distal tubular sections connected by an interconnecting strip; the proximal tubular section comprising a proximal opening, a distal opening, and an inner diameter sufficient to accommodate the distal mandrel portion therein; the distal tubular section comprising a proximal opening and a distal opening, the proximal opening configured to receive the previously introduced wire guide therethrough;
wherein the interconnecting strip extends axially and circumferentially in communication with the distal opening in the first tubular section and the proximal opening in the second tubular section; and
wherein the proximal tubular section is disposed over and adhesively bonded to the first and second mandrel portions.

23. The coupling wire guide of claim 22, wherein the tracking tip comprises a radiopaque marker material.
